(19) United States
(12) Patent Application Publication
(10) Pub. No.: US 2014/0121547 A1
(43) Pub. Date: May 1, 2014

(54) Pulse Sensor Measurement System And Method

(71) Applicants: Evian Gordon, Vaucluse (AU); Peter Maul, Figtree (AU)

(72) Inventors: Evian Gordon, Vaucluse (AU); Peter Maul, Figtree (AU)

(21) Appl. No.: 14/148,327

(22) Filed: Jan. 6, 2014

Related U.S. Application Data
(63) Continuation of application No. PCT/AU2012/000810, filed on Jul. 6, 2012.

(30) Foreign Application Priority Data
Jul. 6, 2011 (AU) 2011902694

(51) Int. Cl. A61B 5/024

(52) U.S. Cl. CPC A61B 5/02444
USPC 600/500

(57) ABSTRACT

A sensor device for transcutaneous monitoring a blood flow within a human or animal, the sensor device including: a microphone for picking up pneumo-acoustic changes; and a diaphragm interposed between the microphone and the surface of a user when in use, forming a sealed cavity between the microphone and diaphragm surface.

### FIELD OF THE INVENTION

The present invention relates to pulse sensors and, in particular, discloses a microphone based pulse sensor for attachment to the wrist or the like for sensing acoustic vibrations.

### BACKGROUND OF THE INVENTION

Any discussion of the background art throughout the specification should in no way be considered as an admission that such art is widely known or forms part of common general knowledge in the field.

In monitoring applications, it is often desirable to monitor the pulse of an animal or human. This can often be achieved by mounting a monitoring device on an external surface near the pulse of the animal or human. Ideally, it is best to ensure the best signal is obtained by a pulse measurement system.

### SUMMARY OF THE INVENTION

It is an object of the present invention to provide an improved pulse sensor head for measuring a pulse.

In accordance with a first aspect of the present invention, there is provided a sensor device for transcutaneous monitoring a blood flow within a human or animal, the sensor device including: a microphone for picking up pneumo-acoustic changes; and a diaphragm interposed between the microphone and the surface of a user when in use, forming a sealed cavity between the microphone and diaphragm surface. When in use, the sealed cavity can be at greater than atmospheric pressure.

The microphone can be mounted on a planar surface and the diaphragm can be also mounted to the planar surface. The diaphragm forms a generally convex surface engaging the human or animal when in use. The diaphragm can be mounted on a surrounding skirt and engages with the planar surface. The skirt limits excessive deformation of the diaphragm in use.

Further, the sensor can include a wrist strap for mounting the sensor on the wrist of a human user.

### BRIEF DESCRIPTION OF THE DRAWINGS

Preferred embodiments of the invention will now be described, by way of example only, with reference to the accompanying drawings in which:

FIG. 1 illustrates a first exploded perspective top view of a pulse sensor constructed in accordance with the present invention;

FIG. 2 illustrates a second exploded perspective bottom view of a pulse sensor constructed in accordance with the present invention; and

FIG. 3 illustrates an operational environment of the preferred embodiment.

### DETAILED DESCRIPTION OF THE INVENTION

In the preferred embodiments there is provided a pulse sensor head for interconnection with a monitor for monitoring the pulse rate on the wrist of a patient. The pulse rate is a proxy for the heart rate of the patient or user. The preferred embodiment provides a transducer placed on the wrist pulse point which acquires the pulse pneumo-acoustically. The resultant electrical signal is than processed with a conventional audio amplifier and then sent to recording or signal processing.

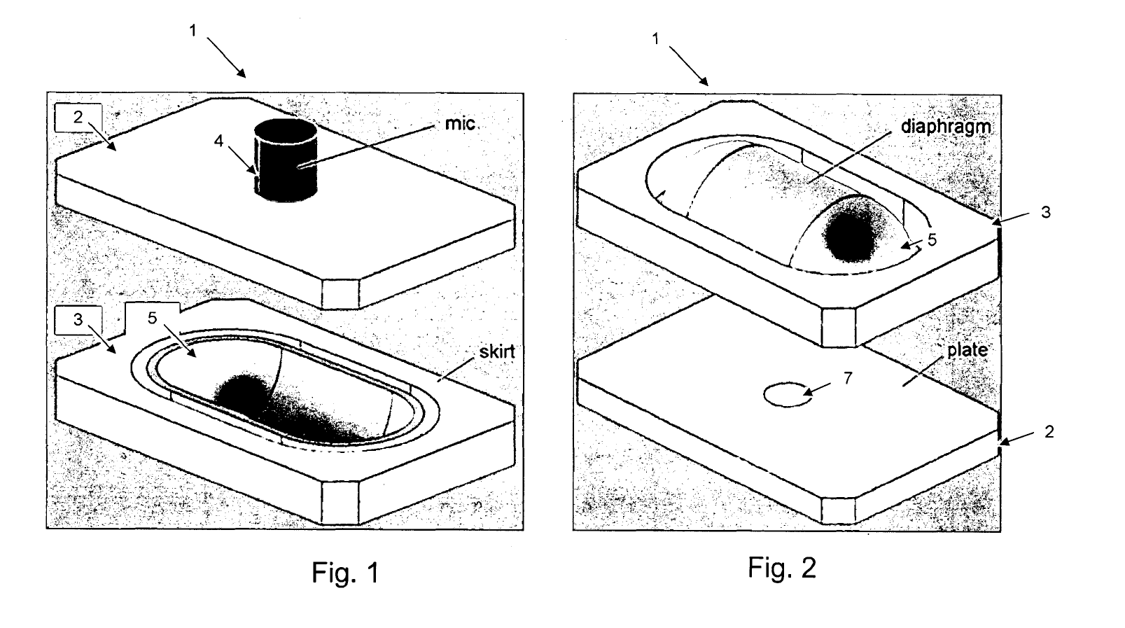

Turning initially to FIG. 1, there is illustrated an exploded side perspective view of the pulse sensor of the preferred embodiment. This sensor includes four main pieces including a plate 2, skirt 3, microphone 4, and diaphragm 5. The plate 2, microphone 4 and diaphragm 5 are all attached together with an air tight seal. The skirt 3 need not be airtight, as it simply provides the diaphragm with a rigid housing and prevents it from compressing excessively.

FIG. 2 illustrates a bottom exploded perspective view and shows the plate 2 having an aperture 7 for the improvement of the microphone pickup.

The diaphragm and plate form an airtight chamber at a pressure of roughly one atmosphere. When strapped to the wrist, the diaphragm extrudes and the slightly elevated pressure compared to atmosphere creates a virtual “finger” that helps convey the pulse signal to the microphone.

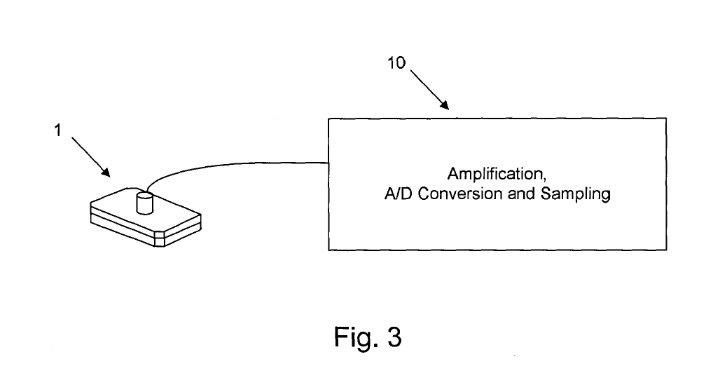

As illustrates in FIG. 3, the signal from the microphone 1 then goes conventionally 10 to amplification, A/D conversion and digital recording and processing.

A benefit of this device is that it is of a compact form and can be worn on the wrist and takes the pulse reading from there directly. Further, it does not require a Bluetoothed chest strap, nor a finger- or earlobe-probe.

### Interpretation

The following description and figures make use of reference numerals to assist the addressee understand the structure and function of the embodiments. Like reference numerals are used in different embodiments to designate features having the same or similar function and/or structure.

The drawings need to be viewed as a whole and together with the associated text in this specification. In particular, some of the drawings selectively omit including all features in all instances to provide greater clarity about the specific features being described. While this is done to assist the reader, it should not be taken that those features are not disclosed or are not required for the operation of the relevant embodiment.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of exemplary embodiments of the invention, various features of the invention are sometimes grouped together in a single embodiment, Fig., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

What is claimed is:

1. A sensor device for transcutaneous monitoring of blood flow within a human or animal, the sensor device including:
a microphone for picking up pneumo-acoustic changes; and
a diaphragm interposed between the microphone and the surface of a user when in use, forming a sealed cavity between the microphone and diaphragm surface.

2. The sensor as claimed in claim 1 wherein said microphone is mounted on a planar surface and said diaphragm is also mounted to said planar surface.

3. The sensor as claimed in claim 2 wherein said diaphragm forms a generally convex surface engaging the human or animal when in use.

4. The sensor as claimed in claim 2 wherein said diaphragm is mounted on a surrounding skirt and engages with the planar surface.

5. The sensor as claimed in claim 2 wherein said skirt limits excessive deformation of said diaphragm in use.

6. The sensor as claimed in claim 1 further including a wrist strap for mounting the sensor on the wrist of a human user.

7. The sensor as claimed in claim 1 wherein, when in use, said sealed cavity is at greater than atmospheric pressure.

8. The sensor as claimed in claim 7 wherein an increase in cavity pressure is consequential upon engagement of the diaphragm with the human or animal being monitored.

9. A sensor device for transcutaneous monitoring a blood flow within a human or animal, substantially as hereinbefore described with reference to the accompanying drawings.
